(19) United States
(12) Patent Application Publication
(10) Pub. No.: US 2015/0004636 A1
(43) Pub. Date: Jan. 1, 2015

(54) NEURONAL BIOSENSOR FOR DETECTION OF TOXINS

(71) Applicant: SAINT LOUIS UNIVERSITY, ST. LOUIS, MO (US)

(72) Inventors: Hannah Kutosky, Granite City, IL (US); Rebecca Kuntz Willits, Akron, OH (US); Amy B. Harkins, St. Louis, MO (US); Shelley Minteer, St. Louis, MO (US)

(73) Assignee: SAINT LOUIS UNIVERSITY, St. Louis, MO (US)

(21) Appl. No.: 13/624,242

(22) Filed: Sep. 21, 2012

Related U.S. Application Data
(60) Provisional application No. 61/537,135, filed on Sep. 21, 2011.

(51) Int. Cl. G01N 33/50; G01N 33/52

(52) U.S. Cl. CPC G01N 33/5014; G01N 33/52; G01N 2458/00
USPC 435/29; 435/288.7

(57) ABSTRACT

A biosensor combines a neuron with an optical sensor to detect toxins in fluids. The present disclosure provides an embodiment for an instrument that can utilize neurons as a biosensor that can provide optical methods to detect and measure low amounts of toxin exposure in humans.

### CROSS-REFERENCE TO RELATED APPLICATIONS

This disclosure claims priority to an earlier filed Provisional Patent application no. 61537135 filed Sep. 21, 2011 entitled Neuronal Bio-sensor for Rapid Detection of Toxins, which is hereby incorporated herein in its entirety.

### BACKGROUND OF THE DISCLOSURE

1. Field of Disclosure

This disclosure relates generally to sensors for detecting toxins, and more particularly, to bio-sensors for detecting environmental toxins and chemical warfare agents.

2. Background Art

Environmental exposure to toxins is currently an issue in military and medical settings. Both children and adults are affected by toxic exposure in the environment which can result from a range of sources, including accidental leakage of insecticides or deliberate gas and chemical warfare. Treatment after toxin exposure is dependent on the type of toxin and the resources available to the medical personnel. Rapid detection of toxin exposure in a patient will help determine how to begin treatment. An instrument is needed that could easily and readily be used in either an emergency room setting or military field setting, even in locations lacking basic hospital needs. There is a need to develop an efficient measure for the detection of exposure to toxins.

In order to detect possible toxins, many different types of toxin sensors have been used for various applications including paper toxin sensors, bioengineered bacteria, and array biosensors. Quick, portable toxin sensors have been created using filter paper and antibodies. These sensors are small and easy to use, but paper toxin sensors are also expensive due to the high price of antibodies.

Other biosensors have been developed to detect hazardous compounds found in gasoline or other petroleum products. Genetically engineered bacteria, called bio-reporters, have been designed to emit light when they come into contact and ingest specific toxins.

Some biosensors are currently being used in the monitoring of chemicals and detection of drugs and chemicals in the body. Array biosensors have been created and used to detect toxins in clinical fluids, environmental samples, and food by utilizing antibodies. Individual toxins have been tested by using an array of different antibodies each specific to a different toxin. However, the lowest possible level of toxins that was detected was 0.5 ng mL-1. Therefore it would be beneficial to develop a biosensor that could detect smaller amounts of toxins that could be present in samples such as blood, saliva, or urine. A more sensitive biosensor could prevent deteriorative effects on the body from occurring before they begin.

### BRIEF SUMMARY OF DISCLOSURE

The present disclosure utilizes neurons as a living biosensor utilizing optical indicators employed to transduce the signal from a biochemical signal to a signal that can be detected by an optical transducer or optical sensor (i.e., CCD camera, photomultiplier tube, etc.) By utilizing the ability of neurons to communicate through changes in calcium concentration and/or vesicle release and fusion, a highly sensitive and time-resolved method for the detection of toxins is the result. Neuronal communication and behavior can be determined through vesicle release and fusion and fluctuation in calcium.

In one aspect, the present disclosure is directed to a biosensor comprising a neuron, wherein the neuronal cell is fluorescently labeled, and an optical sensor positioned to view a cellular activity of the neuron before and after its contact with a molecule in a fluid sample.

In another aspect, the present disclosure is directed to a method of using a biosensor to detect a molecule in a fluid sample comprising the following steps: selecting a neuron from a population of neurons, wherein the neuron responds with a cellular activity to the molecule in the fluid sample; depositing the neuron on a surface; labeling the neuron with a fluorescent nanoparticle; incubating the neuron with the fluid sample; and positioning and focusing an optical sensor to view the cellular activity in the neuron when the neuron is incubating in the fluid sample.

An embodiment of the disclosure can use microscopy and spectroscopy to visualize and measure calcium concentration and/or vesicle recycling in neurons. The present disclosure provides an embodiment for an instrument that can utilize neurons as a biosensor, and that can provide optical methods to detect and measure low amounts of toxin exposure in animals, mammals or humans. In addition, other aspects to neuronal organelles can be stained and used as the fluorescent detector, such as mitochondria and lysosomes. The sensitivity of neurons to toxins will allow detectable measurements of fluorescent signals from the neurons and their organelles.

Biosensors have diverse and important properties that enable them to function as sensors. A biosensor is a device used to detect or monitor biological and/or chemical changes in an environment or organism. There are three main parts of a biosensor: a biological recognition element, a signal transducer, and a response signal, as shown in FIG. 1. Biosensors generally contain three major parts: a biological recognition element, a signal transducer, and a response signal.

Applying neurons to the world of biosensors is beneficial because neurons can be used as a warning of possible toxicity as well as a measure of the toxicity of a substance at a much lower level than non-neuronal biosensors. Currently neurons are being used as biosensors, but the methods used are either invasive or complex, and involve probing the cell body with an electrode or using a microarray of electrical and/or acoustic field detection.

As mentioned above, biosensors can contain three major parts: a biological recognition element, a signal transducer, and a response signal. The biological recognition element can be used to detect an analyte, the biological or chemical element being monitored, in a given biological environment. Once the analyte is detected, the information can be sent to the signal transducer which quantifies the amount of analyte and converts it into an output that can be extracted and analyzed.

A type of biosensors: optical biosensors are divided into two subcategories based on their detection: light absorption and light emission. The former detects the amount of light being absorbed in a reaction and the latter detects the amount of light emitted during a luminescent process. Examples of light absorption optical biosensors are the glucose test strips used by diabetes patients that measure the amount of glucose in the blood. The test strips contain lightly colored chromogen that produces a dye when bound with hydrogen peroxide that is created during aerobic glucose oxidation. Light emission optical biosensors usually use a spectrometer to detect the amount of interference, or absorption of light, created by a chemical or biological interface.

### BRIEF DESCRIPTIONS OF THE DRAWINGS

For a better understanding of the present disclosure, reference may be made to the accompanying drawings in which:

FIG. 1 is an illustration of a schematic of a biosensor;

FIG. 1A is an illustration of one embodiment of a device;

FIG. 2 is an illustration of a neuron;

FIG. 3 is an illustration of a synaptic cleft;

FIG. 4 is an illustration of a synaptic vesicle cycle;

FIG. 5 is an image of Ca²⁺ concentration in neurons with or without KCl;

FIG. 6 is an illustration of waveforms of fluorescence intensity of Ca²⁺ concentration in neurons before and during stimulation;

FIG. 7 is an illustration of concentrations of intracellular Ca²⁺;

FIG. 8A is an illustration of concentrations of intracellular Ca²⁺ of neurons at rest or when treated with glutamate and nicotine;

FIG. 8B is an illustration of concentrations of intracellular Ca²⁺ of neurons at rest or when treated with ionomycin, KCl and boric acid;

FIG. 9 is an illustration of concentrations of intracellular Ca²⁺ for two subgroups of neurons (non-responsive and responsive) treated with Glutamate;

FIG. 10 shows Jablonski Diagram;

FIG. 11 shows peak and emission spectra for fluorophores;

FIG. 12 shows schematic of the internal parts of a spectrofluorometer;

FIG. 13 shows neurons imaged using (A) phase, (B) Cy5, (C) TxRed, (D) Cy3, (E) FITC, and (F) DAPI filters;

FIG. 14 shows neurons stained with FM® 4-64;

FIG. 15 shows neurons stained with FM® 4-64 and treated with ionomycin;

FIG. 16 shows neurons stained with FM® 4-64 and treated with KCl;

FIG. 17 shows neurons stained with FM® 4-64 and treated with boric acid;

FIG. 18 shows neurons stained with FM® 4-64 and treated with glutamate;

FIG. 19 shows neurons stained with FM® 4-64 and treated with nicotine;

FIG. 20 shows emission intensities of neurons (A) unlabeled, (B) stained with FM® 4-64, (C) treated with ionomycin, and (D) post-stimulated FM® 4-64 washed with 1×HBSS;

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

### DETAILED DESCRIPTIONS OF THE DISCLOSURE

In one aspect, the present disclosure is directed to a biosensor comprising a neuron, wherein the neuronal cell is fluorescently labeled, and an optical sensor positioned to view a cellular activity of the neuron before and after its contact with a molecule in a fluid sample. The optical sensor is operable to view signals of the cellular activity consisting of vesicles release, vesicle incorporation, mitochondria integrity, organelle activities, and a combination thereof. In another embodiment, the neuron is selected from a population of neurons, wherein it experiences a cellular activity when in contact with the molecule in the fluid sample. The molecule could be a toxin and the cellular activity is a hallmark of cell death. The neuron could be labeled with a fluorescent nanoparticle; and wherein a change in the fluorescent nanoparticle correlates with the cellular activity. The fluorescent nanoparticle could be a fluorescent calcium indicator or a fluorescent dye. The fluorescent nanoparticle could consist of Fura-2, FM® dye and a combination thereof. The fluorescent dye is associated with a membrane of the neuron. The biosensor technology comprises fluorescent microscopy and/or spectroscopy imaging.

In another aspect, the present disclosure is directed to method of using a biosensor to detect a molecule in a fluid sample comprising the following steps: selecting a neuron from a population of neurons, wherein the neuron responds with a cellular activity to the molecule in the fluid sample; depositing the neuron on a surface; labeling the neuron with a fluorescent nanoparticle; incubating the neuron with the fluid sample; and positioning and focusing an optical sensor to view the cellular activity in the neuron when the neuron is incubating in the fluid sample. Another embodiment is that the molecule is a toxin and the cellular activity is a hallmark of cell death. The cellular activity could be a change in cellular calcium concentration or a change in neuronal membrane. The change in neuronal membrane could consist of the release of membrane vesicles, incorporation of membrane vesicles and combination thereof. The fluorescent nanoparticle could be a fluorescent calcium indicator, such as Fura-2. The fluorescent nanoparticle could be a fluorescent dye, such as a FM® dye. The fluorescent dye is generally associated with a membrane of the neuron.

Another embodiment of the present disclosure comprising a neuron based biosensor having labeled synaptic vesicles to enhance optical detection of neuronal stimulation and recycling vesicles teaches a novel apparatus and method for detecting the presence of toxins in a human specimen.

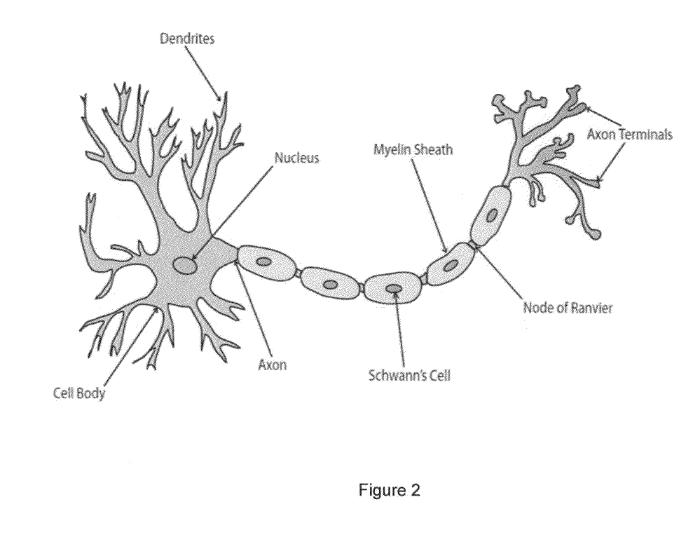

The nervous system is the communication network of the body that sends signals between various parts of the body and the brain. The signals are carried along neurons, which are a major cell type that make up the nervous system. The central nervous system is composed of the brain and the spinal cord, and the peripheral nervous system is comprised of all the other neurons that extend from the spinal cord to nearly every location of the body. The nervous system controls any processes or movements of the body and receives incoming signals from the body's environment and sends them to the brain to be interpreted. FIG. 2 is an illustration of a neuron. The nervous system is the communication network of the body that sends signals between various parts of the body and the brain. The signals are carried along neurons, which are a major cell type that make up the nervous system. Like other cells, a neuron has a nucleus that resides in the cell body. Multiple processes, or dendrites, extend from the cell body, the longest of them being the axon.

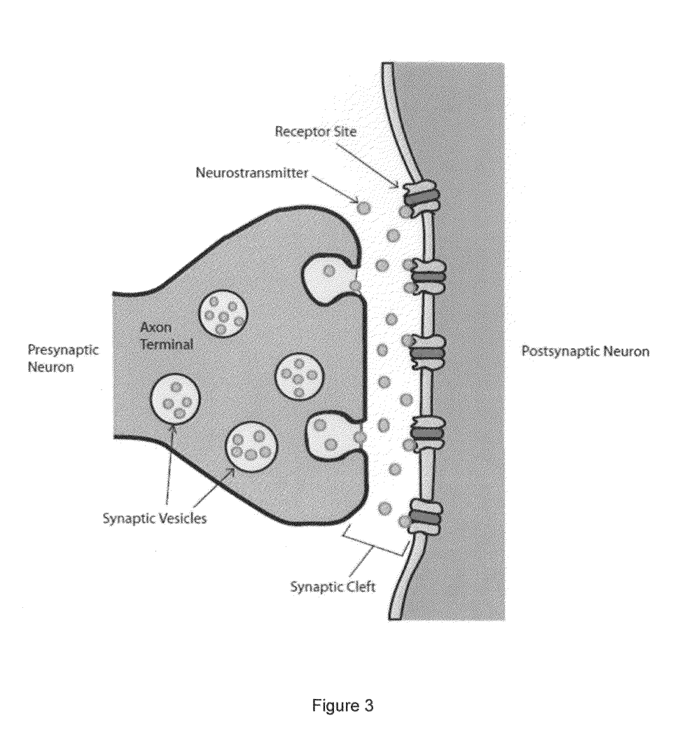

Messages from the cell body travel down the axon to the axon terminals where they are transported via electrical or chemical message to another cell or target tissue. The neuron that releases the message, which is often in the form of a neurotransmitter, is called the presynaptic neuron and the neuron receiving the message is called the postsynaptic neuron. The postsynaptic neuron has receptor sites embedded in the membrane that transduces the neurotransmitter signal to the postsynaptic neuron. See FIG. 3. Once the message, or signal, is received by the postsynaptic neuron, the signal will then travel down the axon of the postsynaptic neuron resulting in a wave of depolarization, called an action potential.

Action potentials are the signals sent down the axon of a neuron and are all-or-nothing events that can be excitatory or inhibitory. Along the length of the axon are myelin sheaths that are small insulations made of fatty substance that enhances the speed of an action potential. The exposed areas of axon between the myelin sheaths, called nodes of Ranvier, are where the ions flow in and out of the axon. Although some neurons communicate only through electrical synapses in which the action potential will travel through specialized channels between neurons called gap junctions, most neurons communicate via chemical synapses. The space between chemically synaptic neurons is called the synaptic cleft, shown in FIG. 3. Action potentials cannot be directly transmitted across the synaptic cleft, and therefore neurotransmitters are released from the presynaptic neuron to activate specialized postsynaptic receptors that bind to the released neurotransmitter on the other side of the synaptic cleft. Excitatory neurotransmitters will depolarize the postsynaptic neuron and inhibitory neurotransmitters will hyperpolarize the postsynaptic neuron. As multiple neuronal inputs are processed by the receiving neuron, the decision to conduct the next action potential or not is determined by the specific strengths and summation of inputs.

FIG. 3 is an illustration of the synaptic cleft. The synaptic cleft is the space between chemically synaptic neurons. Action potentials cannot be directly transmitted across the synaptic cleft, and therefore neurotransmitters are released from the presynaptic neuron and taken up by the postsynaptic neuron on the other side of the synaptic cleft.

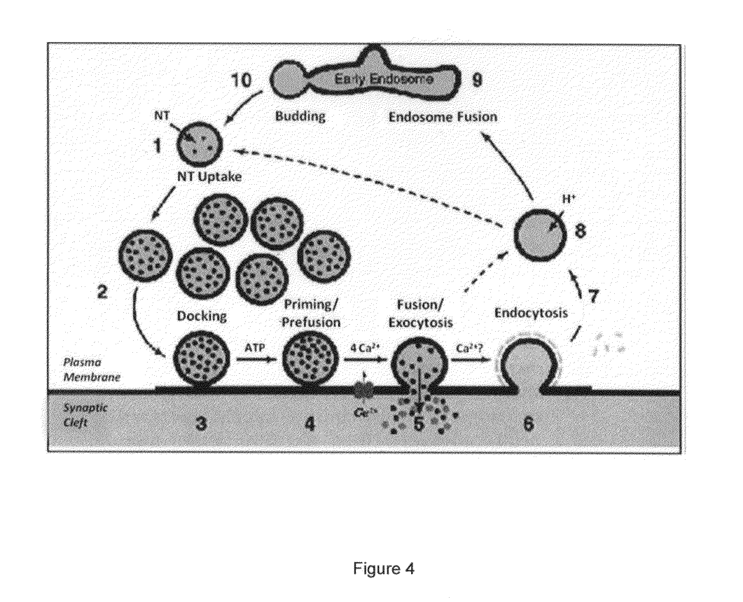

For vesicular packaging of chemical neurotransmitters, neurotransmitters are synthesized and packaged into vesicles both in the cell body and at the axon terminal. The packaging, or uptake, of neurotransmitters is mediated by transporter proteins in the synaptic vesicle membrane that are specific to each type of neurotransmitter. For example, there are separate transport proteins for glutamate, ATP, and acetylcholine. Once filled with neurotransmitters, the synaptic vesicles move to the active zone near the presynaptic plasma membrane where they become attached to the membrane. See FIG. 4. This process is also called docking. Docking only occurs at the active zone where attached vesicles are primed for calcium-dependent release. Studies have shown that partial fusion of the vesicle with the membrane during priming may occur and can be referred to as “kiss and run.” When the vesicle membrane fully fuses with the plasma membrane and loses its integrity as a vesicle, “full fusion” can occur, termed exocytosis. After exocytosis, and before recycling of the membrane, termed endocytosis, the synaptic vesicle is coated with clathrin, a protein layer that helps form the shape of the synaptic vesicle. Endocytosis of the empty synaptic vesicle occurs rapidly following exocytosis. The clathrin coat is then removed from the newly formed vesicle membrane. Synaptic vesicles are recycled into the nerve terminal where they are either refilled with neurotransmitter immediately or undergo fusion with an early endosome and are sorted before returning to the synaptic vesicle cycle.

One embodiment of the disclosure utilizes the natural process of the neuron to configure an effective biosensor to detect the presence of toxins. These natural processes of the neuron are stimulated when there is a presence of a toxin and these natural processes will respond differently on the type of toxin that is present or sensed by the neuron based biosensor. In order to improve the ability to monitor the activity of the neuron, the present disclosure utilizes marking techniques to enhance the ability to optically monitor activity.

After the action potential is transmitted to the axon terminal, depolarization of the presynaptic terminal occurs, permitting an influx of Ca²⁺ ions to enter through voltage dependent Ca²⁺ channels. Ca²⁺ ions trigger fusion of the vesicle membrane with the plasma membrane to release the neurotransmitter into the synaptic cleft. The neurotransmitter then attaches to its designated receptor on the membrane of the postsynaptic neuron. By creating a signal in the postsynaptic neuron, neurotransmitters can either stimulate another action potential or inhibit the neuron from passing on another signal. Vesicles then undergo endocytosis. During this reuptake process it is possible that other materials, such as small fluorescent nanoparticles located extracellularly to the neurons. The recycling of synaptic vesicles, and therefore the fluorescent nanoparticles, can be used to detect stimulation of neurons. The present disclosure uses optical means to detect the recycling of synaptic vesicles and this optical detection and monitoring is enhanced by the labeling.

Another way that neuronal stimulation can be detected is through the membrane recycling that occurs during the synaptic vesicle cycle. Fluorescent membrane labels such as FM® styryl dyes can be used to stain the neuron membrane before stimulation occurs. Immediately following the neurotransmitter release from the stored synaptic vesicles, sections of the fluorescently labeled plasma membrane are endocytosed as part of the re-releasable synaptic vesicle's membrane. The further stimulation of the neurons will recycle sections of the fluorescently labeled membrane and determine the amount of stimulation the neuron is undergoing. Fluorescent nanoparticles and membrane labels can be used to detect and monitor the recycling of synaptic vesicles and therefore the stimulation of neurons.

When neurons are stimulated, intracellular Ca²⁺ increases. This increase in Ca²⁺ can be due to either the influx of extracellular Ca²⁺ or the release of Ca²⁺ from intracellular stores from organelles, such as mitochondria or the sarcoplasmic reticulum. If neurons maintain an increase in intracellular Ca²⁺ for extended periods of time, the cell will undergo programmed cell death, called apoptosis. By tracking the recycling of fluorescently labeled synaptic vesicles, neuronal stimulation and health can be determined.

The present disclosure includes methods for addressing the tracking of the recycling vesicles. One method is to use fluorescent nanoparticles. Multiple types of fluorescent particles can be used to label recycling vesicles in neurons. Fluorescent detectors, such as FM® dyes, can also be used in cell tracking and labeling applications. FM® dyes are lipophilic styryl compounds that are nontoxic to cells, water-soluble, and become intensely fluorescent when attached to the outer surface of cell membranes. Different types of FM® dyes can be used to emit different wavelengths of light. FM® dyes can be used in many applications including membrane labeling and vesicle trafficking, and can be internalized in neuronal recycled synaptic vesicles during active neurotransmitter release. FM® 1-43 can be used to study and label synaptic vesicles involved in clathrin-mediated or bulk endocytosis as well as exocytosis in the Drosophila neuromuscular junction. When measuring synaptic vesicle endocytosis and exocytosis the present disclosure can utilize FM® 1-43 to distinguish different types of vesicular trafficking.

Many biosensors have been developed to detect toxic chemicals and gases in samples such as oil, water supplies, food, and even clinical fluids. The biosensors previously developed provide minimal detection of toxins and are not sensitive enough to detect small amounts of toxins that can be present in humans. Neurons have also previously been used as biosensors; however, invasive methods were utilized such as electrode probing and microarrays of electrical and acoustic field detection. Whereas, the present disclosure uses neurons as a biosensor by labeling the neuron and/or the intracellular vesicles that undergo release during normal neuronal function and/or the intracellular organelles such as mitochondria and lysosomes. The labeling is accomplished by using fluorescent nanoparticles or other fluorescent markers, and with the se of the fluorescent labeling, the effect of toxins on neurons can be detected via optical sensors as the fluorescent signals from the particles and/or labeled neurons are altered. The use of neurons as a biosensor can provide more rapid and sensitive detection of toxins present in either environmental or human samples without the use of invasive measures. By utilizing the inherent sensitivity of neurons, neurons can be used effectively as biosensors.

Methods including fluorescence microscopy and spectroscopy, including Ca²⁺ imaging, can be used to detect and measure the reaction of neurons when exposed to different toxins, thus determining the type and concentration of each toxin. Ca²⁺ imaging can be used for observing the effect each toxin has on the neurons, and microscopy can be used to visualize the recycling of fluorescently-labeled synaptic vesicles in response to the exposure to each toxin. Spectroscopy can be used to measure the change in fluorescence in the neuronal culture caused by the loss of fluorescently labeled synaptic vesicles in the neurons in response to each toxin. The toxic effect on neurons can be detected and measured using three different optical sensors: Ca²⁺ imaging, fluorescence microscopy, and fluorescence spectroscopy. The disclosure can be more suitable than some of the prior methods for the detection and measurement of toxins in humans in an ‘onsite’ and medical setting. With the present disclosure a neuron-based biosensor can detect, measure, and determine the presence, concentration, and type of toxin present in human or environmental samples.

As discussed above, neurons communicate with each other via electrical and chemical synapses. For chemical transmission, neurons release neurotransmitters from vesicles in response to stimulation. The stimulation results in depolarization of the presynaptic terminal membrane to activate voltage dependent Ca²⁺ channels, shown in FIG. 3. These channels in turn open to permit an influx and subsequent increase in intracellular Ca²⁺. As the concentration of intracellular Ca²⁺ increases, Ca²⁺ ions bind to Ca²⁺ receptive proteins, such as synaptotagmin, located on the synaptic vesicles at the active zones. Binding of Ca²⁺ causes the synaptic vesicles to initiate opening of the fusion pore and expansion for release of neurotransmitters from the lumen of the vesicle. The rise of intracellular Ca²⁺ with stimulation in neurons can be, for example, measured using a fluorescent Ca²⁺ indicator such as Fura-2. The binding of Ca²⁺ to the Ca²⁺ indicator can be imaged during stimulation to determine the effects that potentially stimulating and toxic solutions have on intracellular Ca²⁺. An increase of intracellular Ca²⁺ at the terminals of the neurons are directly related to the Ca²⁺ dependent vesicle secretion and therefore, to the recycling events for vesicles.

Using an imaging technique such as Fura-2 imaging, whole fields of neurons can be rapidly screened for increases of intracellular Ca²⁺ that would affect vesicle recycling. Therefore, this method can be used as an initial screen for potential toxins, stimulating solutions and controls in the dorsal root ganglia (DRG) neurons. Fura-2AM is a membrane permeable molecule that consists of a ratiometric free acid Ca²⁺ indicator dye called Fura-2 and acetoxymethyl (AM) ester. Fura-2AM is readily permeable to cell membranes, and once inside a neuron, intracellular esterases will cleave the ester and rap the free acid form of Fura-2 within the neuronal membrane. This trapped Fura-2 is used to measure intracellular Ca²⁺ concentrations in cells. Fura-2 binds to Ca²⁺ ions in a 1:1 ratio of dye to ion. It uses the ratio of two sets of fluorescence excitation/emission wavelengths that change depending on whether the dye is bound to Ca²⁺ or not. By exciting Fura-2 at 340 nm and 380 nm, and collecting emitted light at 510 nm wavelength of light, two spectra are collected. The ratio of the spectra can be calculated and converted to a value of intracellular Ca²⁺ concentration by a calibration curve. Advantages of using a ratiometric indicator such as Fura-2 include the reduction of error that can be caused by uneven cell thickness, uneven dye loading, and leakage of dye. Toxic effects of photobleaching are also eliminated. The ratio of emission intensities can be used to calculate the concentration of intracellular Ca²⁺ in many different cell types, including neurons.

Neuron can be readily stimulated with low concentrations of chemicals, stimulants, and neurotoxins such as nicotine, potassium chloride (KCl), ionomycin, and glutamate. Glutamate is a major excitatory neurotransmitter in the central nervous system that activates N-methyl D-aspartate (NMDA) receptors and causes Ca²⁺ to enter the cell. Glutamate is known as a neurotoxin that becomes toxic when it accumulates in the extracellular space in the brain causing hypoxia and ischemia, believed to be due to prolonged elevation of intracellular Ca²⁺ levels. Glutamate has induced toxic effects on multiple cell types including retinal, cortical, cerebellar, and hippocampal neurons. Ionomycin is a selective Ca²⁺ ionophore that carries Ca²⁺ across plasma membranes if it is present in the solution surrounding the cells. Nicotine activates the nicotinic acetylcholine receptors in neurons and causes an increase of intracellular Ca²⁺ by Ca²⁺ influx, similar to ionomycin. Extracellular KCl causes an increase in the plasma membrane potential and depolarization of the cell. Upon stimulation with either stimulants or toxins, neuronal membranes are depolarized and will activate the voltage dependent Ca²⁺ channels. This results in an increase of intracellular Ca²⁺ that is proportional to the degree of stimulation caused by either the stimulants or toxins. Fura-2 can be used to monitor and report the signal of increase of intracellular Ca²⁺ by Ca²⁺ imaging in neurons.

A common source for neurons is chick embryos, especially for the extraction of peripheral ganglia. The extraction of neurons from chick embryos has multiple advantages. Chick peripheral ganglia can be extracted using a simple and inexpensive preparation that can be performed daily; the chick eggs can be stored in a refrigerator prior to incubation and incubated to ensure a constant supply of neurons. Chick peripheral neurons can grow in low densities allowing for single cell experimentation. They can also withstand experimental handling such as being outside an incubator for extended periods of time.

In one embodiment dorsal root ganglia (DRG) are useful in neuronal biosensors because their neurons begin to form axons within 1-3 hours after plating and can be manipulated by several neurotrophic factors including neural growth factor (NGF). DRGs, like many peripheral ganglia, can be purified by removing the other cells (glial cells and fibroblasts) from their culture. Because of ease in preparation and the experimental advantages, DRG neurons can be used for the present disclosure. By measuring the concentration of intracellular Ca²⁺ in DRG neurons with imaging of Fura-2, the effects of different chemicals on neurons can be determined.

TABLE 1
1x HBSS

| Salt | Molecular Weight (g/mol) | Concentration (mM) |
|---|---|---|
| NaCl | 58.44 | 137 |
| KCl | 74.55 | 5.4 |
| MgCl₂—6H₂O | 203.3 | 0.5 |
| MgSO₄—7H₂O | 246.5 | 0.4 |
| KH₂PO₄ | 136.1 | 0.44 |
| Na₂HPO₄—7H₂O | 268.1 | 0.34 |

TABLE 2
50 mM KCl

| Salt | Molecular Weight (g/mol) | Concentration (mM) |
|---|---|---|
| NaCl | 58.44 | 87 |
| KCl | 74.55 | 5.4 |
| MgCl₂—6H₂O | 203.3 | 1 |
| CaCl₂ dihydrate | 147.0 | 5 |
| HEPES | 238.3 | 12 |
| D(+)Glucose | 180.2 | 10 |

Solutions can be mixed with 18 MΩ ultra-purified H₂O in the above order, pH of 7.4 for HBSS and 7.35 for KCl. The osmolality can be matched for solutions (290 310 mOsm) by adding sucrose when needed.

Specific-Pathogen-Free (S.P.F.) fertile chick eggs were ordered for experimentation to prove the concept of the disclosure, its efficacy and application. The eggs were incubated for 10 days before dissection in a dark egg incubator with motion at a dry temperature of 86-100° F. and wet temperature of 84-86° F. From the ventral side, the spines of the embryos were exposed, the kidneys were removed one at a time under higher dissecting power carefully so as not to remove the nerve chains situated just below the kidney along the ventral side of the spinal cord. The DRGs were each dissected from the area between the bottom of the ribs and the posterior end of the embryo, placed in a tissue culture dish containing F12 media. DRGs from 12 embryos were transferred with a fire-polished Louis Pasteur pipette to a sterile 15 ml tube. After the DRGs settled to the bottom of the tube, a pipette was used to carefully remove the F12 media, avoiding removal of any DRGs. Ten mLs of 5% trypsin were added to the tube with DRGs, mixed, and incubated for 30 minutes in a 37° C. water bath. Every 10 minutes of incubation the tube was removed from the water bath and tapped to mix the DRGs and allow the trypsin to digest connective tissue to separate the neurons. After incubation, trypsin was removed using a pipette. The cells were rinsed with 2 ml of Neurobasal media 2-3 times by adding media, allowing the DRGs to settle to the bottom of the tube before removing the media, and adding another 2 ml of media. After the final rinse, the volume was brought up to 1 ml and the dissociated neurons were triturated with a medium-sized, then a small-sized, fire-polished Louis Pasteur pipette until the cell solution appeared cloudy and no individual DRGs were present in the media. A small amount of the solution was transferred to a hemocytometer and the single cells were counted. The cell solution was diluted so that 10-15×10⁴ cells were added to an Alconox® treated, 0.1 mg/ml collagen coated, 25 mm diameter round glass coverslip in a 35 mm dish with 2 ml of Neurobasal media. The DRGs were incubated in 5% CO₂ at 37° C. for 2-3 days before experimentation. Approximately 1 mL of media was replaced with new media every other day until experiments began.

Experiments began 2-3 days after neurons were plated. A dish of neurons was washed with 1×HBSS then 1×HBSS+1 mg/ml bovine serum albumin (BSA) before the cells were bathed in 4 μM Fura-2AM in 1×HBSS+BSA for incubation for 45 min at 37° C. in 5% CO₂. The cells were washed 4× with 1×HBSS and incubated for another 20-60 min in 1×HBSS. A coverslip containing the neurons was removed after the final wash and placed in an apparatus for calcium imaging.

All toxins including nicotine, boric acid, ionomycin, glutamate, and glycine were prepared in 1×HBSS according to their final concentrations (Table 3).

TABLE 3
Toxins used in Fura-2 imaging experiments.

| | Stock | | Final |
|---|---|---|---|
| Toxin | Concentration | Solvent | Concentration |
| Boric Acid | 61.83 g/mol, dry | 1X HBSS | 50 mM |
| Glutamate | 88.2 mM | 1X HBSS | 100 μM, 500 μM, 1 mM |
| Ionomycin | 2 mM in DMSO | 1X HBSS | ~100 μM |
| Nicotine | 462.41 g/mol, dry | 1X HBSS | 100 μM, 300 μM |

The coverslip containing the neurons was placed in a perfusion apparatus onto an Olympus IX70 microscope equipped with a 20× fluorescence objective, a Mercury lamp and filter sets that optimized the Fura-2 excitation (340 and 389 nm) and emission wavelengths (510 nm). The cells were bathed continuously by perfusion of solutions at ˜4.7 mL/min. The cells were bathed with 1×HBSS during software setup and for resting Ca²⁺ measurements before exposure to stimulating or toxic solutions. InCyt Im2™ software was used to measure the emission intensities and convert to Ca²⁺ concentrations. Only designated areas of each neuron soma were chosen for measurements. Calibration curves were generated for conversion of light intensity emitted per excitation wavelength using standard Ca²⁺ solutions in the free acid form of Fura-2 to convert emitted light intensity to Ca²⁺ concentration. Each experiment began with 3 min wash with HBSS to establish resting intracellular Ca²⁺ measurements. Depending on the toxin used, the neurons were then exposed to a flow of toxic/stimulating solution for 5-10 min. Each experiment ended with a 1 min. wash of HBSS to determine reversal or change of the stimulating/toxic solution.

The concentration of intracellular Ca²⁺ was graphed versus time in InCyt software. The intensity of emitted light at both 340 and 389 nm wavelengths was recorded as well as the ratio of F340/F38 and the converted concentration of Ca²⁺ at each measurement point acquired every 10 sec. Data from each dish were exported to Origin 8 to plot the mean±standard error of the mean (SEM) of the Ca²⁺ concentration as a function of time for the experimental stimulations. The averages of all cells exposed to the same stimulant/toxin were plotted as bar graphs. Individual neurons that did not respond similarly to the majority of the neurons in each group were eliminated from the group, except for neurons stimulated with glutamate. Cells stimulated with glutamate were analyzed twice; once with all the data and once in a separate set analysis.

Except where noted, a Kruskal-Wallis ANOVA was performed with Tukey post hoc test to determine the level of significance between data (OriginPro 8, Copyright © 1991-2009 OriginLab Corporation). When noted, a paired sample Wilcoxon signed rank test was performed on smaller paired sample sizes.

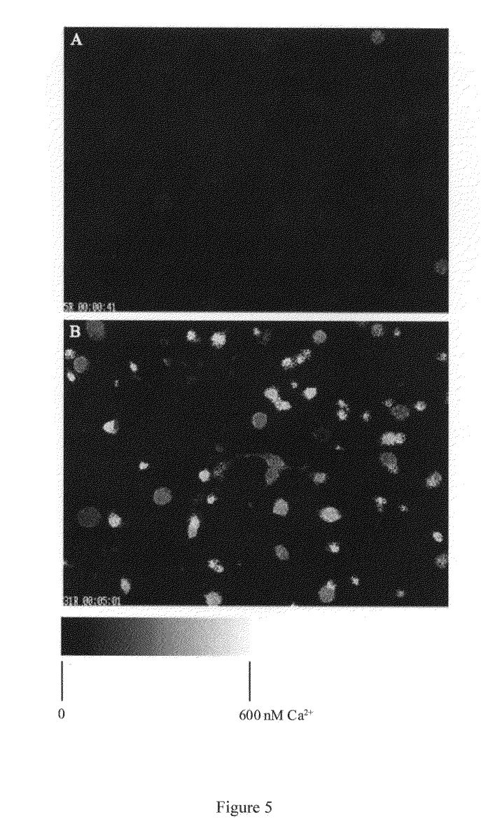

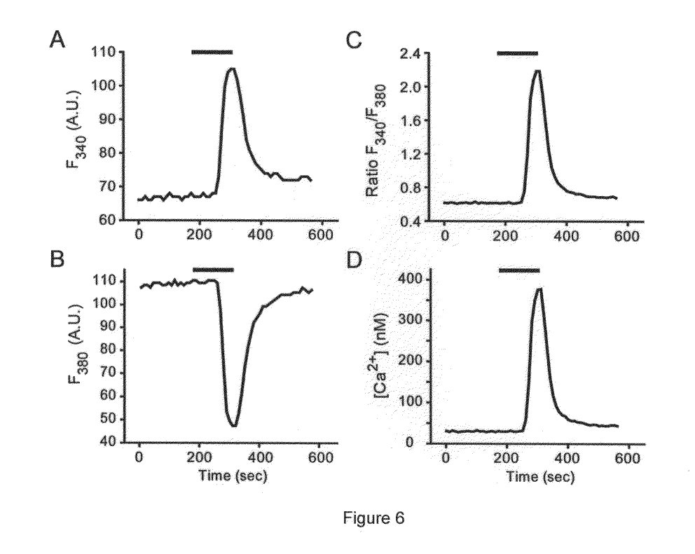

Upon examination of the test result it can be determined that when the cells are at rest, dim outlines of the neurons are apparent, as the Ca²⁺ concentration is low (typically <100 nM). However, shortly after application of the stimulating solution, in this case, 50 mM KCl, the neurons responded by depolarizing and allowing extracellular Ca²⁺ to enter the cell. This Ca²⁺ influx is depicted as white shown in FIG. 5. From these images, regions of interest are acquired per dish of neurons, and from each of the regions, a value is acquired for the fluorescence intensity at 340 nm and 380 nm (F340 and F380, respectively). These values are plotted per region of interest versus time as shown by examples in FIGS. 6A and B. The ratio of F340/F380 is calculated (FIG. 6C), converted to a plot of Ca²⁺ concentration versus time (FIG. 6D).

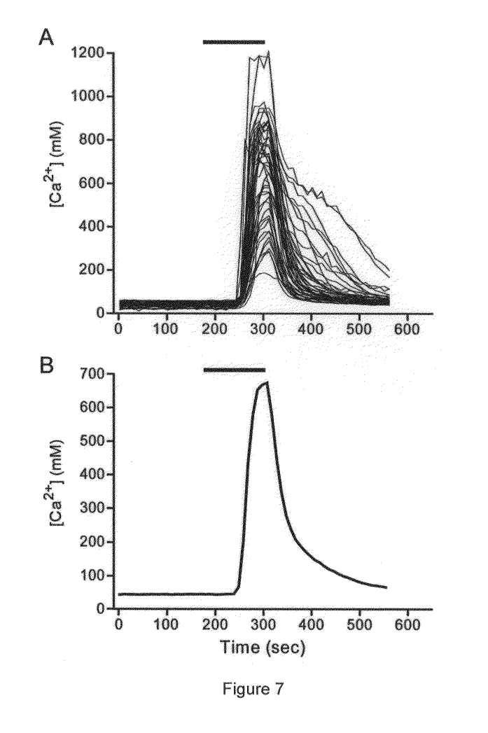

The intracellular Ca²⁺ concentration corresponds to depolarization of the cell. Influx of Ca²⁺ will trigger release of synaptic vesicles, as well as potentially trigger additional Ca²⁺ dependent processes including secondary signaling pathways and cell death. Therefore, it is anticipated that the extent of intracellular Ca²⁺ influx is a monitor for stimulation and/or cell death. FIG. 6: Waveforms of the fluorescence intensity at (A) 340 nm and (B) 380 nm, (C) the ratio of the fluorescence intensity at 340 nm/380 nm, and (D) the concentration of intracellular Ca²⁺ in DRG neurons during stimulation (bar) with 50 mM KCl. From the imaged field of neurons, many regions of interest were acquired simultaneously and plotted as Ca²⁺ concentration versus time (FIG. 7A), and averaged (FIG. 7B). The solid bar above the waveform corresponds to time the cells were exposed to the stimulant, in this case, 50 mM KCl. The example in FIG. 7 shows the response of all the cells, each to a different extent, to the stimulation by KCl.

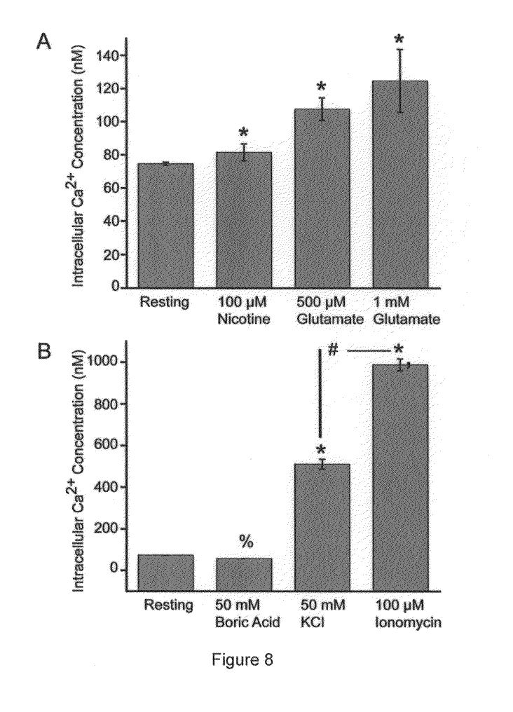

However, when glutamate was applied, some cells clearly responded to the stimulant, while others did not. Therefore, the data acquired with glutamate as a stimulant were analyzed separately as two different populations as well as an averaged population to statistically compare data from the ‘responding” versus ‘non-responding’ populations of cell. Resting Ca²⁺ was measured from the average of the first 180 sec of HBSS prior to switching to a stimulating or toxic solution. The resting Ca²⁺ for all of the cells, regardless of stimulating solutions, was averaged shown in FIGS. 8A & 8B (left column), and compared to the change in intracellular Ca²⁺ with stimulation (FIGS. 8A and 8B). On average, resting Ca²⁺ was 75 nM (±1 nM; n=974). FIG. 7: Concentrations of intracellular Ca²⁺ (A) for each neuron measure from one dish and (B) the mean intracellular Ca²⁺ concentration of the DRG neuronal population during stimulation (bar) with 50 mM KCl. FIGS. 8A and 8B: The mean intracellular Ca²⁺ concentration (±standard error of the mean) of individual DRG neurons at rest and during stimulation. A Kruskal Wallis ANOVA and a Tukey post hoc test determined difference from resting (*p<0.01), from ionomycin and KCl (+p<0.05), and between sets of data (#p<0.05). A Paired Sample Wilcoxon Signed Rank Test determined differences between cells before and after exposure to boric acid (% p<0.05).

The averaged change in intracellular Ca²⁺ in response to stimulants/toxins compared to the average resting Ca²⁺ is shown in FIGS. 8A and 8B. The time points at which the levels of intracellular Ca²⁺ were the highest in response to the stimulation were averaged and collected as the mean (±standard error of the mean) Ca²⁺ concentration for each set of data. Because ionomycin is a Ca²⁺ ionophore and used as a positive control, influx of Ca²⁺ evoked a large and irreversible concentration of intracellular Ca²⁺, on average 988 nM (±29 nM; n=228), compared to resting Ca²⁺ (*p<0.01). When depolarized with 50 mM KCl, the influx of Ca²⁺ was greater, on average 511 nM (±24 nM; n=104), than resting Ca²⁺ (*p<0.01) as well as significantly less than the influx of Ca²⁺ observed with ionomycin (#p<0.05). Both ionomycin and KCl produced a greater increase in intracellular Ca²⁺ compared to the other stimulants and compared to resting Ca²⁺ (+p<0.05). Stimulating with 1 mM glutamate, 500 μM glutamate, and 100 μM nicotine resulted in increases in Ca²⁺ concentration greater than resting Ca²⁺ (*p<0.01), but with average values less than 200 nM. Boric acid was used as a negative control, as it is not known to be toxic to mammalian cells, and was similar to resting Ca²⁺. The mean intracellular Ca²⁺ concentration of neurons exposed to 50 mM boric acid was significantly less, on average 58 nM (±1 nM; n=175) (% p<0.05 using a Paired Sample Wilcoxon Signed Rank Test) than the mean resting concentration of those neurons exposed only to boric acid, 71 nM (±2 nM).

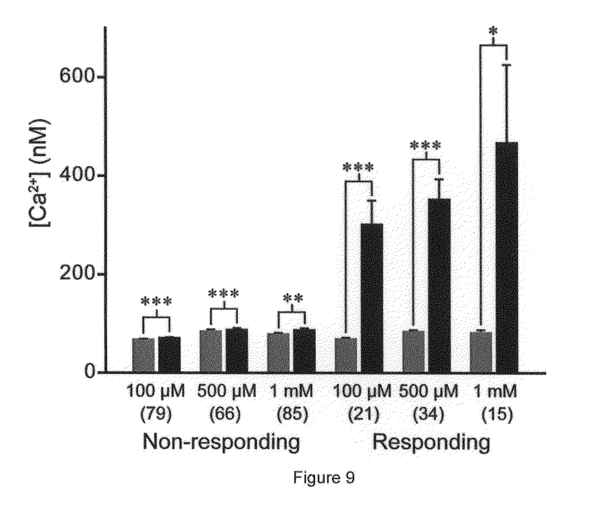

Neurons responded in two ways to application of glutamate as described above. Cells either had an increase in Ca²⁺ levels when stimulated with glutamate or did not respond at all. Therefore, the cells that responded were considered separately from those that did not respond using a paired sample Wilcoxon signed rank test. The intracellular Ca²⁺ concentrations of both sets of neurons that did and did not respond to glutamate are shown in FIG. 9 for each of the three concentrations of glutamate compared to their average resting Ca²⁺ (gray bars). The influx of Ca²⁺ in both non responding and responding cells was higher during stimulation than at rest with average intracellular Ca²⁺ concentrations of 71 nM (±1.4 nM; n=100) for 100 μM with a resting Ca²⁺ of 67 nM (±1.5 nM), 88 nM (±3.2 nM; n=146) for 500 μM with a resting Ca²⁺ of 84 nM (±3.5 nM), and 87 nM (±3.4 nM; n=38) for 1 mM glutamate with a resting Ca²⁺ of 79 nM (±2.3 nM) for non-responding cells and 301 nM (±48.6 nM, n=27) for 100 μM with resting of Ca²⁺68.7 nM (±2.6 nM), 352 nM (±41.4 nM; n=75) for 500 μM with a resting Ca²⁺ of 82 nm (±5.4 nM) in responding cells. Below each of the cell groups stimulated with different concentrations of glutamate, the percent of non-responding or responding cells is shown. FIG. 9: The mean intracellular Ca²⁺ concentrations (±standard error of the mean) of (A) non-responding and (B) responding DRGs at rest (gray) and during stimulation (black) with 100 μM, 500 μM, and 1 mM glutamate. A Paired Sample Wilcoxon Signed Rank Test determined the differences shown (*p<0.05, **p<0.01, ***p<0.001).

It is clear from the above test results that neurons utilized as biosensors can be effective. It is also clear that the responses when exposed to toxin can be readily discerned, measured and characterized. Further, it is clear that different toxins cause different responses depending on the type and amount of toxin and the different responses are distinguishable.

In one embodiment of the present disclosure, imaging with Fura-2 can be an accurate way to measure the concentration of calcium within a region of interest in cell samples. Because it uses the ratio of emitted light from a Ca²⁺ indicator molecule, excitation at two different wavelengths allows quick measurements of Ca²⁺ concentration. A 1:1 ratio of Ca²⁺ ions binding of Fura-2 allows for a simple conversion to Ca²⁺ concentration for individual regions of interest, such as cells. Because neurons experience an influx of Ca²⁺ when stimulate or under distress, a change of intracellular Ca²⁺ is an easy and accurate way to measure the effects of different stimulants and neurotoxins on neurons. As state in the results and shown in FIG. 8B, ionomycin causes an influx of Ca²⁺ into the neurons compared to resting neurons. As seen in FIG. 8A, KCl, 1 mM glutamate, 500 μM glutamate, and 100 μM nicotine caused an increase in intracellular Ca²⁺ compared to the neurons at rest. Ionomycin and KCl stimulated the neurons more than any other stimulating/toxic condition.

When comparing the population of cells that were only exposed to boric acid, boric acid statistically decreased the concentration of intracellular Ca²⁺ compared to the Ca²⁺ concentrations of those same neurons at rest. When the populations of neurons stimulated with glutamate were analyzed separately as non-responding and responding cells (FIG. 9), each concentration of glutamate caused an increase of intracellular Ca²⁺ compared to resting cells. The controls used for the Fura-2 experiments, boric acid and ionomycin, caused changes in intracellular Ca²⁺ that were expected. Since ionomycin is a strong Ca²⁺ ionophore, it is highly toxic to neurons and causes an irreversible increase in intracellular Ca²⁺. Boric acid, not known to be toxic to mammalian neurons, caused a slight, but significant, decrease of intracellular Ca²⁺ in neurons.

The increase of intracellular Ca²⁺ caused by KCl was expected because KCl is commonly used to rapidly and reversibly depolarize neurons. The resulting increases of intracellular Ca²⁺ in response to glutamate and nicotine are statistically significant when compared to resting, but do not show as great an increase as KCl and ionomycin. It is well known that nicotine causes depolarization in neurons via the nicotinic acetylcholine receptors present on the cell membrane. Glutamate has also been classified as a neurotoxin that binds to NMDA receptors and causes an influx of extracellular Ca²⁺ in neurons. This data showed a significant but small increase in intracellular Ca²⁺ in neurons exposed to 100 μM nicotine and various concentrations of glutamate. Nicotine and glutamate may have increased the intracellular Ca²⁺ concentration to a lesser extent than ionomycin and KCl due to the stage of development of the DRGs when they are extracted. Chick peripheral DRGs may not develop nicotinic acetylcholine or NMDA receptors until a later stage of development, in this case past day 10 incubation.

Future studies that would clarify this issue include dissection of chick DRGs at various points of embryonic chick development and measuring changes in intracellular Ca²⁺ resulting from nicotine and glutamate. From the data obtained during Ca²⁺ imaging, several stimulants/toxins cause a significant change of intracellular Ca²⁺ in chick DRG neurons. The amount of Ca²⁺ influx into the neurons causes proportional amounts of vesicles to be recycled in the neurons. By staining neuronal vesicles and/or membranes, vesicle recycling can be tracked, visualized, and measured using live imaging fluorescence microscopy and fluorescence spectroscopy.

The above results verify the efficacy of the present disclosure, which utilizes neurons as biosensors. One embodiment of the present disclosure combines neurons as a biosensor with optical indicators as a source of measurement. By utilizing the ability of neurons to communicate through vesicle release and fusion, a highly sensitive and time-resolved method for the detection of toxins is the result. Neuron communication and behavior can be determined through vesicle release and fusion. The present disclosure can use microscopy and spectroscopy to visualize and measure vesicle recycling in neurons, respectively. The present disclosure provides an embodiment for an instrument that can utilize neurons as a biosensor, and that can provide optical methods to detect and measure low amounts of toxin exposure in humans.

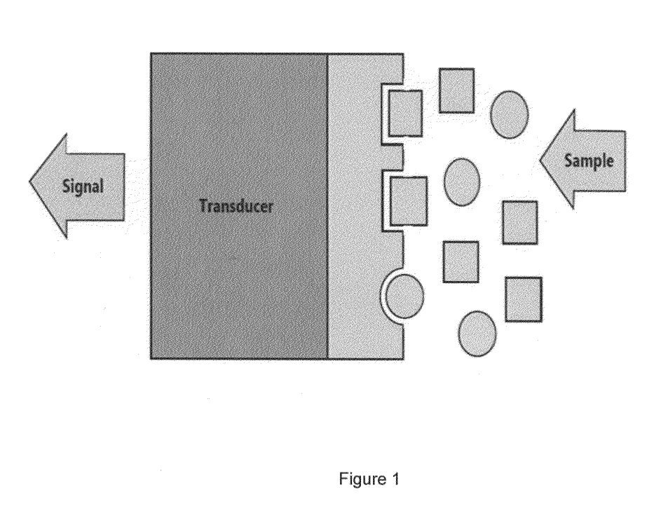

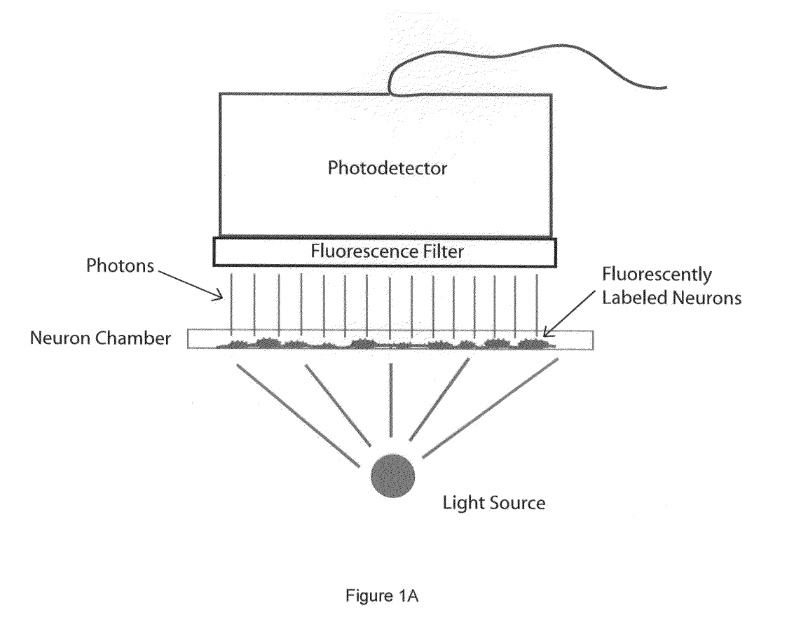

One embodiment of a device of the present disclosure is illustrated in FIG. 1A.

As discussed above, the present disclosure can use microscopy and spectroscopy to visualize and measure vesicle recycling in neurons, respectively. The present disclosure provides an embodiment for an instrument that can utilize neurons as a biosensor, and that can provide optical methods to detect and measure low amounts of toxin exposure in human. Microscopy can be used as one embodiment of the present disclosure.

One embodiment of the present disclosure proposes the use of optical microscopy.

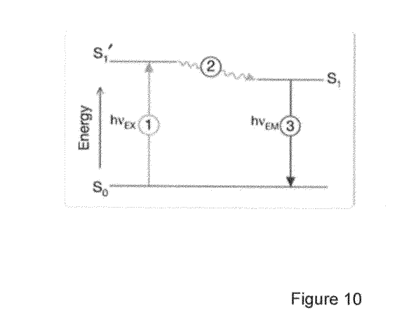

Fluorescence is defined by the light emitted by a fluorophore that has absorbed light at lower wavelength. The fluorescence cycle, shown in FIG. 10, consists of three stages: excitation, transient excited lifetime, and emission (fluorescence). During excitation a fluorophore in the ground energy state (S₀) absorbs energy from photons (hvex) and enters a high-energy state (S₁) which is determined by the wavelength of the exciting light. The fluorophore will lose some of its energy during the transiently excited lifetime stage and fluoresce during the emission stage, losing all of its energy and returning to its ground energy state. Some fluorophores can repeat the fluorescence cycle multiple times before they become photobleached. FIG. 10: Jablonski Diagram. The fluorescence cycle consists of three stages: excitation, transient excited lifetime, and emission (fluorescence). A single fluorophore can go through the fluorescence cycle several times before becoming photobleached. One embodiment of the present disclosure proposes using the characteristics of fluorophores and fluorescence.
Excitation: S₀+hvₑₓ→S₁
Emission: S₁→S₀+hvₑₘ+heat

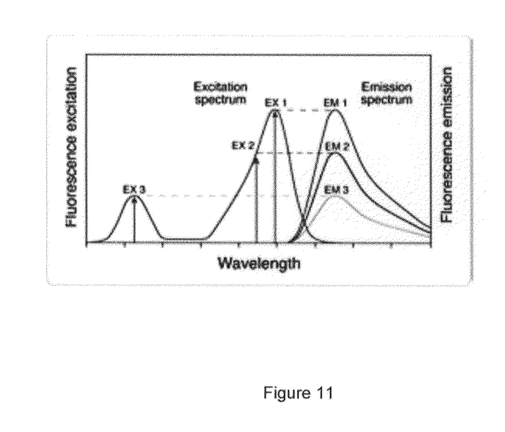

During the emission stage, heat is released due to molecules colliding with the fluorophore. In the equation, h is Planck's constant, vₑₓ is the frequency of the excitation light, and vₑₘ is the frequency of the emitted light. The absorbed, or excitation, light has a shorter wavelength, higher energy and higher frequency. Because energy is lost during the transiently excited lifetime, the fluorescent, or emitted, light has a longer wavelength, lower energy, and lower frequency. Every fluorophore has a characteristic excitation and emission range. The excitation range determine the possible excited states (S₁) a fluorophore can achieve and the emission range represents the wavelengths of light that are emitted from the fluorophore (FIG. 11). When excitation occurs at the excitation maximum, the fluorophore can emit several wavelengths of light, each corresponding to the various energy levels it achieves during the transient excited lifetime. When excitation occurs at a wavelength lower or higher than the excitation maximum wavelength, the fluorophore will emit a lower intensity of light, not a different wavelength of light, as shown in FIG. 11. The shift from the excitation and emission maximum wavelengths is called the Stokes shift and is due to the energy loss in the transient excited lifetime stage of the fluorescence cycle. FIG. 11: Peak excitation and emission spectra. Fluorophores each have a maximum wavelength at which they can be excited, which will result in the maximum amount of emitted light, or fluorescence. If the fluorophore is excited at a different wavelength of light than its excitation maximum it will emit a less intense light, but always at the same wavelength.

Fluorescence has been used in many chemical and biological applications, and is commonly used in microscopy to visualize stained cells and their structures as well as specific proteins. Limitations of fluorescence imaging include structural instability and photobleaching of the fluorophores, but if used properly, fluorescence can provide a very sensitive method of labeling and detection. Fluorescent nanoparticles, such as FM® styryl dyes, can be used in neurons to label and track synaptic vesicle. With the present disclosure, by tracking synaptic vesicles with fluorescence, the extent of neuronal stimulation can be observed using microscopy.

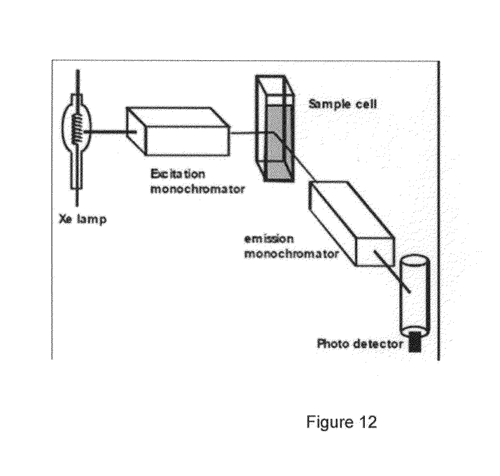

Fluorescence spectroscopy is a method used to quantify fluorescence from a sample after excitation with specific wavelengths of light. Spectrofluorometers contain monochromators to control both excitation and emission wavelength. There are two types of fluorometers: filter fluorometers and spectrofluorometers. Filter fluorometers use two filters, one to isolate an excitation wavelength and the other to isolate an emission wavelength. Filter fluorometers excite and detect fluorescence at small wavelength ranges, limiting their use in specific applications. However, spectrofluorometers use diffraction grating monochromators, or filters, that isolate one excitation wavelength and can detect a wider range of emission wavelengths. Both have the same setup shown in FIG. 12. The photo detector is set up 90 degrees from the light source to reduce interference from the excitation light, which is typically much higher in intensity than the emitted light. Spectrofluorometry is used to measure the fluorescent intensity of FM® labeled neurons before and after exposure to stimulants and toxins in order to prove the efficacy of the present disclosure. FIG. 12: A schematic of the internal parts of a spectrofluorometer. The Xe Lamp is in line with the excitation monochromator (filter) and sample, and the photo detector and emission monochromator are oriented at a 90° angle to reduce interference from the excitation light. For one embodiment of the present disclosure where neurons can be used as a biosensor, an effective form of fluorescence measurement can be used. With the addition of image analysis pre- and post-toxin exposure, images of fluorescently labeled neurons can determine the amount and type of toxin by measuring the amount of fluorescence in neuron samples. An alternative method for neuronal toxicity measurement for the present disclosure is to quantify a change in the fluorescence spectroscopy. Measuring the amount of toxins via change in fluorescence of neurons using fluorescence spectroscopy provides a more portable and affordable option in the design of a neuronal biosensor. Data demonstrating the efficacy of this embodiment is presented below.

The present disclosure can use microscopy and spectroscopy to visualize and measure vesicle recycling in neurons, respectively. The present disclosure provides an embodiment for an instrument that can utilize neurons as a biosensor, and that can provide optical methods to detect and measure low amounts of toxin exposure in humans. The efficacy of the present disclosure's use of fluorescently labeled neurons to determine the amount and type of toxin by measuring the amount of fluorescence in neuron samples is evidenced by the data presented below.

The materials utilized for the testing is as follows: Dow Corning 732 multi-purpose sealant (Cat # NC9917246) and Saint-Gobain Tygon tubing (Cat #141691W) was purchased from Fisher Scientific, Hampton, N.H. 03842, USA. FM® 4-64 styryl dye (Cat # T13320) was purchased from Invitrogen, Carlsbad, Calif. 92008, USA. Further details are in Table 4-1. Thermanox plastic cell culture coverslips (10.5×22 mm, Cat #174934) were purchased from Nalge Nunc International, Rochester, N.Y. 14625, USA. One model CHH-1 coverslip holder was purchased from C&L Instruments, Inc., Hummelstown, Pa. 17036, USA. Clear 4-sided poly(methyl methacrylate) (PMMA) cuvettes (Cat #14-377-015) were purchased from Fisher Scientific, Pittsburgh, Pa. 15275, USA.

TABLE 4
A list of dyes used during microscopy experiments.

| | Stock | | Final |
|---|---|---|---|
| | Concen- | | Concen- |
| Dye | tration | Solvent | tration |
| Dextran, 3 kDa TxRed | 6.7 mM | 50 mM KCl | 100 μM |
| FM ® Styryl Dye, FM ® 4-64 | 1.5 nM | 1X HBSS | 30 μM |
| Qdots, 605 nm | 8 μM | 50 mM KCl | 560 nM |

For microscopy experiments, holes were drilled in the bottom of 35 mm tissue culture dishes with a 7/32″ drill bit and drill press. Etching was performed on 12 mm round glass coverslips that were submerged in 300 ml of boiling 18 MΩ H₂O containing 7.5 g Alconox detergent for 30 minutes. The coverslips were then rinsed with 18MΩ H₂O and dried in a 150 mm Petri dish in a laminar flow hood. A section of Tygon tubing (0.5 inch long, 0.5 inch inner diameter, and 0.75 inch outer diameter) was used to apply Dow Corning 732 multi-purpose sealant to the bottom of the tissue culture dishes just around the hole that was drilled previously. Forceps were used to press one round glass coverslip to the bottom of each tissue culture dish until a continuous seal was formed around the hole. The tissue culture dishes were laid in a fume hood to dry. Exposure to ultraviolet light for at least an hour or treatment with ethanol for approximately 30 seconds was used to sterilize the dishes. Type VII collagen, to promote cell adhesion to the glass coverslip, was added to each dish for coating and allowed to air dry in the laminar flow hood to maintain sterility. Once the dishes were dry, the lids were placed back on the dishes and the edges of the Petri dishes were then wrapped in Parafilm and stored in a 4° C. refrigerator before use.

Focusing on the FM® dye, the dye was thawed at room temperature and vortexed. A 30 μM FM® dye solution was made with 1×HBSS. The solution was heated to 37° C. and vortexed just before each cell staining experiment. The cells were exposed to the FM® dye solution for 5-10 min before imaging.

The media was removed from the 35 mm tissue culture dish using a micropipette. The surface was then rinsed once with 1 ml HBSS. To stimulate the uptake in the neurons, ˜50-70 μl of fluorescent dye solution was added to the well inside the 35 mm dish since this area of the dish is where the imaging occurs. The solution bathe the neurons for approximately 5 min before the dish was rinsed three times with 1 ml of HBSS each rinse. After the final rinse with HBSS, 1 ml of HBSS was added to the dish to prevent drying out during imaging.

At least 30 min before imaging, the humidity, temperature, and floating table controls of the chamber around the microscope were turned on to 5% CO₂ and 37° C. The images were acquired using a Hamamatsu Digital CCD Camera and an Olympus IX81 Motorized Inverted Research Microscope with a Cy5 or TxRed and phase filter, with a 100× oil objective. Three images of each region of interest was acquired with Slidebook 4.1.0.16 and saved: phase alone, Cy5 or TxRed alone, and phase and Cy5 or TxRed combined.

For spectroscopy experiments, DRG neurons were grown on 11×22 mm plastic coverslips that were treated with Alconox and coated with Type VII rat tail collagen, using the same methods that were mentioned previously. Cells were plated with ˜15×10⁴ cells in each 35 mm tissue culture dish containing one plastic coverslip. The cells were incubated in 5% CO₂ at 37° C. for 2 days before experiments began.

Before scanning the cell samples, the coverslip was mounted into a coverglass holder and was held inside a cuvette at a 45° angle from the light source and the photo detector. The coverglass holder was placed in a cuvette containing 1×HBSS to rinse the media from the cells prior to the experiments. For the spectroscopy experiments, the coverglass holder was placed in the cuvette that was filled with either 1×HBSS or stimulating solutions. The orientation of the coverglass in the cuvette was situated so that the cell surface was facing the light source and photodetector, both at 45° angles. For experiments using FM® 4-64, scan were conducted using a HORIBA Jobin Yvon FluoroLog®-3 spectrofluorometer with an excitation wavelength of 558 nm and an emission range was detected from 566 to 750 nm. The slit width for excitation and emission was 3 nm and data was collected every 0.2 sec, once for each wavelength. Fluorescence intensity was detected in counts per second (cps). The data was transferred into MS Excel and analyzed using Matlab.

In Matlab, the maximum intensity of each scan was found for the emission wavelength range of interest and the ratio of the maximum emission intensities was taken between non-labeled, non-stimulated cells and FM®-labeled cells before and after stimulation. This ratio was used to determine the difference in fluorescence, and therefore the amount of stained membranes present in the sample before and after stimulation with ionomycin.

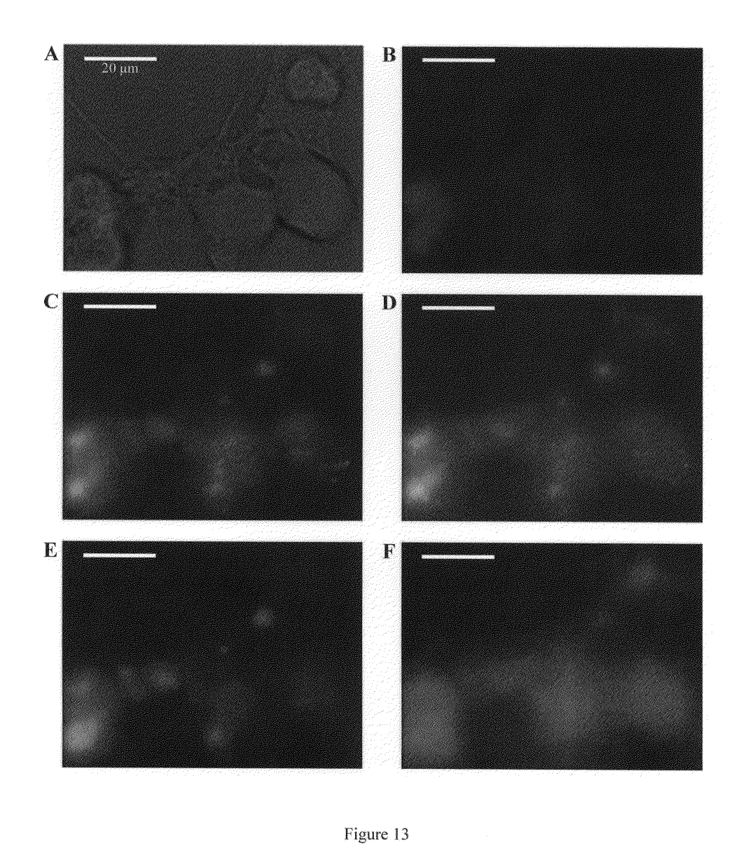

Images were taken of DRG neurons while being bathed with 1×HBSS to test the intensity of endogenous (background) fluorescence. Images taken using DAPI (461 nm emission), FITC (521 nm emission) and Cy3 (570 nm emission) filters showed the most intense endogenous fluorescence while TxRed (615 nm emission) and Cy5 (670 nm emission) showed less (FIG. 13). The images were taken at exposure times in the range of 1-2 sec. FIG. 13: DRG neurons imaged using (A) phase, (B) Cy5, (C) TxRed, (D) Cy3, (E) FITC, and (F) DAPI filters.

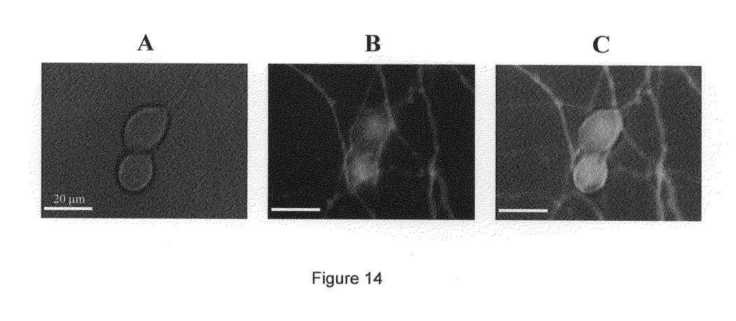

Because FM® dyes bind readily to plasma membranes, FM® 4-64, a fluorescent membrane label, was mixed in 1×HBSS instead of KCl to label the membranes of the neurons. The images show complete staining of the cell membranes around the soma and axons (FIG. 14). FM® 4-64 was chosen to stain the DRG neurons for further tests with stimulants and toxins. FIG. 14: DRG neurons stained with FM® 4-64 and imaged using (A) phase and (B) TxRed filters, and (C) both images merged.

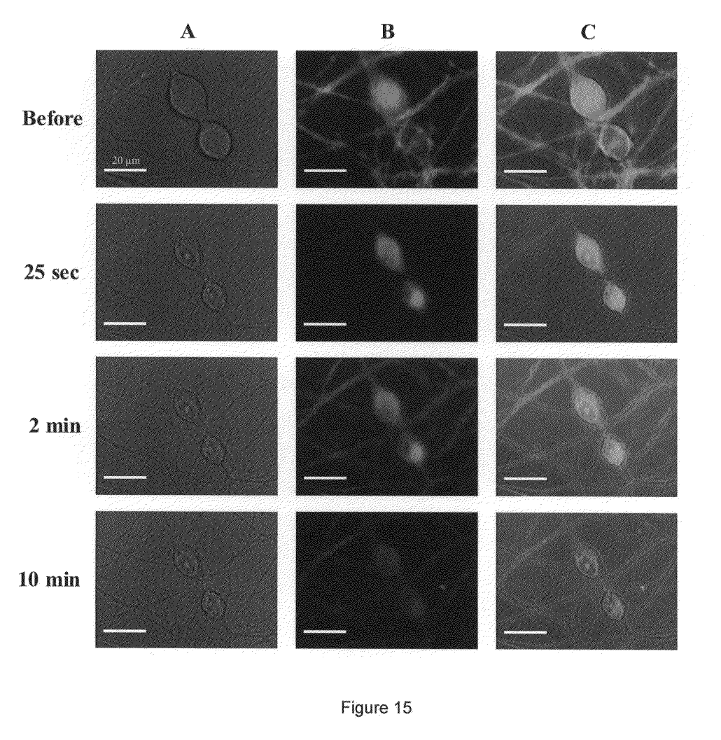

DRG neurons stained with FM® 4-64 were imaged before exposure to 100 μM ionomycin. Without moving the dish from its position, the same cells were imaged every 30-60 sec for 10 min post ionomycin application. The dramatic differences in the membrane stain are shown in FIG. 15 at 25 sec, 2 min, and 10 min after application of ionomycin, compared to the images prior to ionomycin stimulation. Within the first 25 sec of stimulation, the FM® dye migrated from the membranes of the axons into the soma, and after several minutes, the dye was redistributed back into the axons, where it remained for the duration of the experiment. Images acquired before stimulation showed healthy DRG neurons and the phase images during stimulation showed shrinkage of the cell soma. The TxRed image after 10 min of stimulation shows more definite particles of FM® dye whereas the dye was more evenly distributed around the membranes prior to the addition of ionomycin. Over the 10 min of imaging, the samples were moving out of focus and the focus was readjusted intermittently for more consistent images. FIG. 15: DRG neurons stained with FM® 4-64 and imaged using (A) phase and (B) TxRed filters, and (C) both images merged, before and at 25 sec, 2 min, and 10 min after stimulation with 100 μM ionomycin.

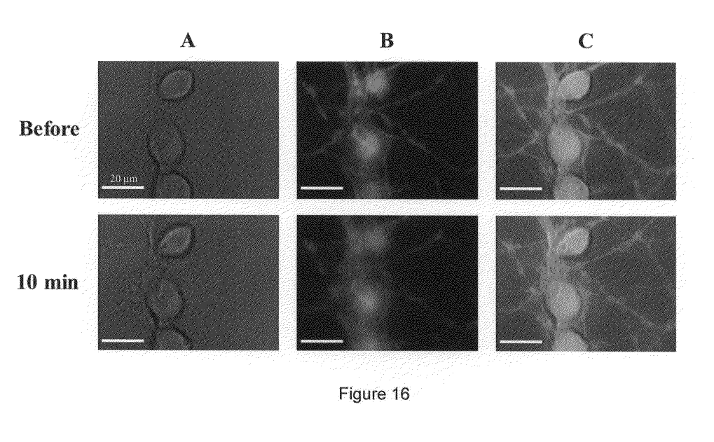

Similar experiments were performed for other stimulants/toxins in DRG neurons labeled with FM® 4-64. For DRG neurons stimulated with 50 mM KCl, images are shown before and 10 min after the addition of KCl (FIG. 16). The images show some migration of FM® dye throughout the DRG neurons. The phase image of FIG. 16 before stimulation shows healthy DRG neurons prior to imaging, but after FM® 4-64 staining, the phase image at 10 min of stimulation shows some swelling in small parts of axons. FIG. 16: DRG neurons stained with FM® 4-64 and imaged using (A) phase and (B) TxRed filters, and (C) both images merged, before and at 10 min after stimulation with 50 mM KCl.

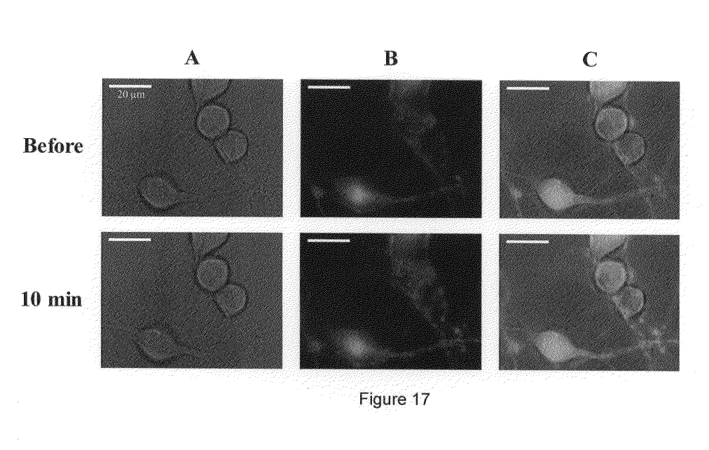

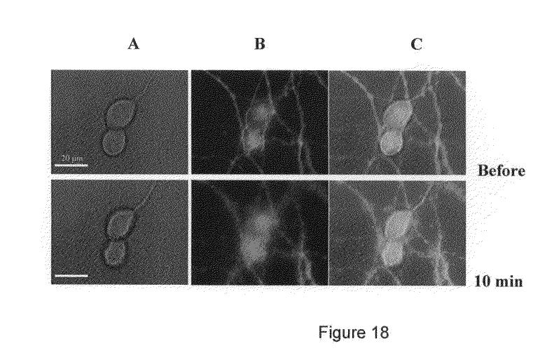

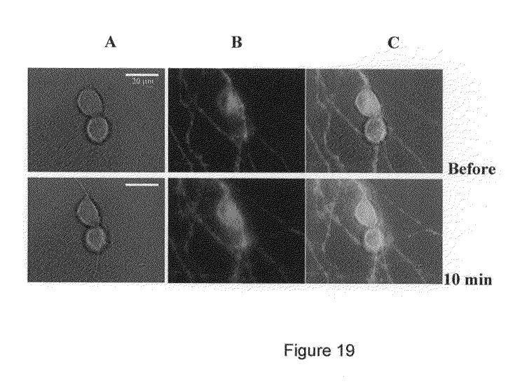

DRG neurons were labeled with FM® 4-64 and each sample was stimulated with 50 mM boric acid (FIG. 17), 500 μM glutamate (FIG. 18), or 100 μM nicotine (FIG. 19). Similar results as obtained with 50 mM KCL occurred by 10 min of stimulation with swollen axons. However, unlike the ionomycin application, there was no migration of FM® 4-64 throughout the cells. Nonetheless, noticeable differences on the membrane are seen comparing the before and after treatment. Similar differences in the membrane structure are seen for neurons treated with glutamate and nicotine. On the other hand, very little differences to no differences are seen for boric acid treated neurons. FIG. 17: DRG neurons stained with FM® 4-64 and imaged using (A) phase and (B) TxRed filters, and (C) both images merged, before and at 10 min after stimulation with 50 mM boric acid. FIG. 18: DRG neurons stained with FM® 4-64 and imaged using (A) phase and (B) TxRed filters, and (C) both image merged, before and at 10 min after stimulation with 500 μM glutamate. FIG. 19: DRG neurons stained with FM® 4-64 and imaged using (A) phase and (B) TxRed filters, and (C) both image merged, before and at 10 min after stimulation with 100 μM nicotine.

To determine whether a change in fluorescence of FM®-labeled neurons could be measured, the cells were stained and stimulated using the same methods mentioned above. Four emission scans were performed: a control scan of the non-labeled neurons in HBSS, a scan of the FM®-labeled neurons in HBSS, a scan of the FM® labeled neurons in ionomycin 5 min after initial stimulation, and a scan of the post stimulated, FM®-labeled neurons in 1×HBSS wash. The cells were rinsed with warm 1×HBSS before each scan, and the cells were stimulated for a total of 10 min before rinsed with 1×HBSS for the fourth scan. The ratio of fluorescence intensities of non-labeled neurons to FM®-labeled neurons, FM®-labeled ionomycin-stimulated neurons, and FM®-labeled ionomycin-stimulated rinsed neurons in Table 5.

TABLE 5
The ratio of peak fluorescence intensities of
scans compared to non-labeled DRG neurons.

| | | Fluorescence | | |
|---|---|---|---|---|
| | Peak | Intensity at | Fluorescence | Ratio of |
| | Emission | Peak Emission | Intensity | Fluorescence |
| | Wavelength | Wavelength | at 667 nm | Intensities at |
| Scan | (nm) | (cps) | (cps) | 667 nm |
| Non-labeled | 667 | 1608 | 1608 | 1 |
| Neurons | | | | |
| FM ®- | 674 | 3098 | 2975 | 1.85 |
| labeled | | | | |
| Neurons | | | | |
| 5 min in | 684 | 3719 | 3303 | 2.05 |
| Ionomycin | | | | |
| Rinsed | 676 | 2362 | 2238 | 1.39 |
| in HBSS | | | | |

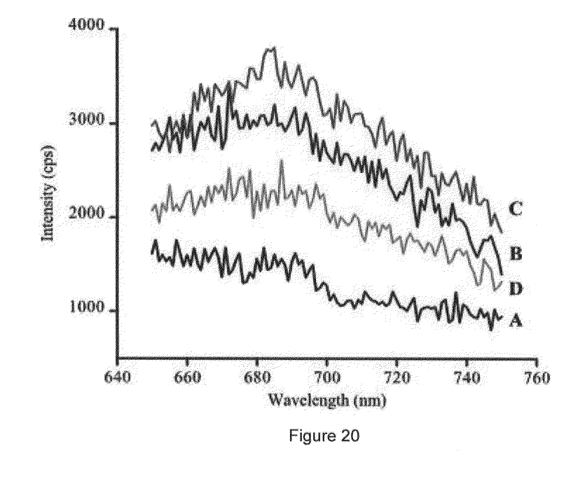

The result of the spectroscopy scans showed that the fluorescence intensity is increased when the cells are labeled with FM® 4-64 and increases even more after 5 min of bathing in ionomycin. However, after 10 min of ionomycin stimulation and a wash with 1×HBSS, the cells become less fluorescent. Waveforms of the scans are shown in FIG. 20. FIG. 20: Emission intensities from 650 to 750 nm of (A) non-labeled neurons, (B) FM®-labeled neurons, (C) neurons after 5 min of stimulation in ionomycin, and (D) poststimulated, FM®-labeled neurons in 1×HBSS wash.

Microscopy is a very useful method used in almost every field of scientific study. By using a live imaging microscope, the behavior of cell samples can be observed while under different conditions. Combining fluorescence with live imaging provides the opportunity to visualize the physical and chemical changes of cells during toxic threats or stimulation. Because they can grow as single cells, individual DRG neurons can be observed while being exposed to toxins/stimulants. The endogenous fluorescence of DRG neurons was detected by imaging with several different fluorescent filters, each for different sets of excitation and emission wavelengths (FIG. 20).

Fluorophores were chosen in the fluorescence ranges that the DRG neurons endogenously emitted the least amount of light to reduce the wavelength interference during microscopy. In this case, the orange to red wavelengths (˜600-670 nm) emitted the least amount in the cells and therefore 605 nm Qdots, red dextrans, and FM® 4-64 were chosen as the fluorescent labels for the stimulant/toxin experiments. The endogenous fluorescence of cells are only known to cause interference at longer exposure times of 400-500 ms, and in this case shorter exposure times (50-150 ms) caused enough excitation of the fluorescent labels chosen. However, labels that emit red wavelengths were still chosen to ensure the least interference, especially when considering future experiments involving measurement of fluorescence intensity.

FM® 4-64 labeled the DRG neurons every time it was applied with 1×HBSS (FIG. 14). Because of its consistent and complete labeling of the neurons, FM® 4-64 was used to test the effect of stimulants and toxins on the calls. The other potential toxins, glutamate (FIG. 18) and nicotine (FIG. 19), the negative control, boric acid (FIG. 17), and the known stimulant, KCl (FIG. 16) was observed.

Ionomycin was anticipated to have a large effect on the DRG neurons based on previous data obtained from Ca²⁺ imaging, and the pattern of migration suggests that the FM® dye was taken up into the cell via synaptic transmission as a result of the massive influx of Ca²⁺. Upon cell death, the FM® dye was redistributed out into the cell extensions and eventually into the extracellular space, thus reducing the amount of fluorescence inside the cell. Experiments using FM® 4-64 as a membrane label, and ionomycin as a positive stimulatory/toxic control has demonstrated the ability of neurons to be used as biosensors.

Further studies were conducted to quantify the amount of fluorescence present in the neurons, thus measuring the extent of neuronal stimulation associated with the specific concentration of the insulting toxin. Spectroscopy is a very useful method in measuring fluorescence, and has been used with chemical or organic solutions, as well as living cells. The data collected in this section shows the preliminary data towards future development. The spectroscopy scans measured an average maximum fluorescence intensity emitted from neurons alone and neurons labeled with FM® 4-64, as predicted. The fluorescence intensity of neurons during 5 min of ionomycin stimulation was larger than the FM®-labeled neurons, possibly due to fluorescence of the ionomycin solution.

Comparing the FM″-labeled neurons (pre-stimulation) to the neurons that were rinsed after stimulation, an expected decrease in fluorescence intensity occurred, suggesting that the FM® dye was ejected from the cells, similar to past studies. The shift in peak emission wavelengths seems to be a result of the change in conditions; ionomycin and FM® 4-64 shifted the peak emission wavelengths up ˜15-19 nm and ˜7-9 nm from non-labeled neuron scans, respectively. Shifts in fluorescence spectra can be caused by a variety of factors including the polarity of the solvent, pH, and the localized concentration of fluorophores. The shift in peak fluorescence that occurred during ionomycin stimulation may be due to the polarity of the stock ionomycin solution that contained dimethyl sulfoxide (DMSO), a highly polar solvent. Regardless of the peak emission shifts, a change in fluorescence intensity at one wavelength was detected and measured.

By using neurons as a biological recognition element in a biosensor, acute traces of biological and chemical agents can be detected. In one embodiment of the present disclosure Ca²⁺ imaging can be used to determine the degree of neuronal stimulation caused by known and potential toxin, and the same chemicals can be applied to neurons during fluorescence microscopy and spectroscopy to visualize and measure the extent of neuronal stimulation via synaptic vesicle recycling of fluorescent nanoparticles, respectively.

By utilizing the Ca²⁺ ionophore ionomycin coupled with Ca²⁺ measurements using Fura-2, increases in intracellular Ca²⁺ were measured in DRG neurons. Both ionomycin and KCl can cause statistically different intracellular Ca²⁺ increases. Various increases of intracellular Ca²⁺ occurred with different concentrations of glutamate and nicotine. When neurons were exposed to glutamate, the majority of the cells, when averaged together, (79%, 66%, and 85% with 100 μM, 500 μM, and 1 mM of glutamate, respectively) did not experience an obvious increase of intracellular Ca²⁺. Only when the individual cells that responded (21%, 34%, and 15% to 100 μM, 500 μM, and 1 mM of glutamate, respectively) with an increase of intracellular Ca²⁺ were analyzed separately from cells that did not respond to glutamate was there a statistical difference between the samples. The same trend followed during stimulation with 100 μM nicotine, with only a few cells experiencing an increase in intracellular Ca²⁺. The reasons for these trends may be due to lack of development of glutamate and nicotinic acetylcholine receptors on day 10 of embryonic chick incubation. Future directions may use chick DRG neurons isolated at a later point in development, such as day 12 or 13 incubation to determine whether the receptor expression has increased, and hence detect a greater increase of intracellular Ca²⁺ when stimulated with either glutamate or nicotine.

To better visualize the physical and chemical process of neuronal stimulation corresponding to an increase in intracellular Ca²⁺, fluorescence microscopy of FM®-labeled neurons was used to image the behavior of the entire cell in response to the chemicals tested in calcium imaging. Fluorescent probes have successfully been used to trace neuronal pathways and determine the health of live neurons in previous studies. Because synaptic transmission only occurs during cell stimulation, fluorescently-labeled cell membranes were used to track the movement of synaptic vesicles during exposure to the stimulants/toxins. According to calcium imaging results, ionomycin causes the most neuronal stimulation, which was confirmed with fluorescence microscopy images.

To improve both the health and the stability of the neurons on the coverslips used in spectroscopy, another option includes growing the neurons on a different coating or material. Instead of Type VII collagen on plastic coverslips, future experiments could be performed with glass coverslips and/or other coatings such as Type I collagen, laminin, or poly-L-lysine to promote adhesion of neurons to the coverslips. Other future work with spectroscopy may include the use of other fluorescent probes, such as a mitochondrial dye, and better design alternatives than a vertical coverslip in the cuvette. The use of an enclosed, stable environment will allow neurons to be healthy and will promote cell adhesion to the surface by reducing the shear forces on the neurons. The future work for this project will be to design a biosensor device that will detect and measure levels of neurotoxin in human samples. The device should have a compartment that fits a neuronal cell culture slide, similar to the Nunc™ membrane. The detection component of the device will either use spectroscopy or a photograph system combined with analysis of images pre- and post-sample insertion. The future of this project will be directed towards designing and building a biosensor using neurons and fluorescence that will allow for ease of use and accessibility to all medical and ‘on-site’ situations for the early detection and measurement of toxin exposure in humans, thus preventing or reducing human mortality associated with environmental toxin exposure.

The various neuronal biosensor embodiments shown above illustrate an apparatus and method for utilizing neuronal biosensors to detect toxins. A user of the present disclosure may choose any of the above embodiments, or an equivalent thereof, depending upon the desired application. In this regard, it is recognized that various forms of the subject neuronal biosensor disclosure could be utilized without departing from the spirit and scope of the present disclosure.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

Other aspects, objects and advantages of the present disclosure can be obtained from a study of the drawings, the disclosure and the appended claims.

The claims are the following:

1. A biosensor comprising:
a neuron, wherein the neuronal cell is fluorescently labeled; and
an optical sensor positioned to view a cellular activity of the neuron before and after its contact with a molecule in a fluid sample.

2. The biosensor of claim 1, wherein the optical sensor is operable to view signals of the cellular activity consisting of vesicles release, vesicle incorporation, mitochondria integrity, organelle activities, and a combination thereof.

3. The biosensor of claim 1, wherein the neuron is selected from a population of neurons; wherein it experiences the cellular activity when in contact with the molecule in the fluid sample.

4. The biosensor of claim 1, wherein the molecule is a toxin and the cellular activity is a hallmark of cell death.

5. The biosensor of claim 1, wherein the neuron is labeled with a fluorescent nanoparticle; and wherein a change in the fluorescent nanoparticle correlates with the cellular activity.

6. The biosensor of claim 5, wherein the fluorescent nanoparticle consists of a fluorescent calcium indicator, fluorescent dye, and a combination thereof.

7. The biosensor of claim 5, wherein the fluorescent nanoparticle consists of Fura-2, FM® dye, and a combination thereof.

8. The biosensor of claim 6, wherein the fluorescent dye is associated with a membrane of the neuron.

9. The biosensor of claim 1, wherein it comprises fluorescent microscopy.

10. The biosensor of claim 1, wherein it comprises spectroscopy imaging.

11. A method of using a biosensor to detect a molecule in a fluid sample comprising the following steps:
selecting a neuron from a population of neurons, wherein the neuron responds with a cellular activity to the molecule in the fluid sample;
depositing the neuron on a surface;
labeling the neuron with a fluorescent nanoparticle;
incubating the neuron with the fluid sample; and
positioning and focusing an optical sensor to view the cellular activity in the neuron when the neuron is incubating in the fluid sample.

12. The method of claim 11, wherein the molecule is a toxin and the cellular activity is a hallmark of cell death.

13. The method of claim 11, wherein the cellular activity is a change in cellular calcium concentration.

14. The method of claim 11, wherein the cellular activity is a change in neuronal membrane.

15. The method of claim 14, wherein the change in neuronal membrane consists of the release of membrane vesicles, incorporation of membrane vesicles and combination thereof.

16. The method of claim 11, wherein the fluorescent nanoparticle is a fluorescent calcium indicator.

17. The method of claim 16, wherein the fluorescent calcium indicator is a Fura-2.

18. The method of claim 11, wherein the fluorescent nanoparticle is a fluorescent dye.

19. The method of claim 18, wherein the fluorescent dye is a FM® dye.

20. The method of claim 18, wherein the fluorescent dye is associated with a membrane of the neuron.
